(19) United States
(12) Patent Application Publication
(10) Pub. No.: US 2020/0000725 A1
(43) Pub. Date: Jan. 2, 2020

(54) Antibiotic Beads for Treatment of an Infection

(71) Applicant: Celanese EVA Performance Polymers Corporation, Irving, TX (US)

(72) Inventors: Christian Schneider, Hattersheim (DE); Melinda Hoskins, Syracuse, IN (US)

(21) Appl. No.: 16/456,417

(22) Filed: Jun. 28, 2019

Related U.S. Application Data
(60) Provisional application No. 62/693,123, filed on Jul. 2, 2018.

(51) Int. Cl. A61K 9/16; A61K 31/7036; A61K 38/14; A61K 31/65; A61K 9/00

(52) U.S. Cl. CPC A61K 9/1635; A61K 31/7036; A61K 9/1694; A61K 31/65; A61K 9/0024; A61K 38/14

(57) ABSTRACT

An antibiotic bead having a size of from about 0.5 to about 20 millimeter is provided. The bead comprises an antibiotic agent dispersed within a polymer matrix, the polymer matrix containing a semi-crystalline olefin copolymer.

### RELATED APPLICATIONS

The present application claims priority to U.S. Application Ser. No. 62/693,123 (filed on Jul. 2, 2018), which is incorporated herein in its entirety by reference thereto.

### BACKGROUND OF THE INVENTION

Infectious wounds are commonly treated by surgical debridement of an area of tissue ischemia within the body. For instance, the standard therapy for treating chronic bone infection (osteomyelitis) includes debridement and sequestrectomy of infected bone, which is then followed by several weeks of intravenous antibiotics. Unfortunately, this treatment has several drawbacks. For example, multiple doses of antibiotics are typically needed, which can become quite expensive. In addition, the intravenous route of administration does not allow the antibiotic to be specifically directed to the location of the infection. Intravenous administration of antibiotics also requires an operation for placement of a catheter, which can lead to serious complications. As a result, various alternatives have been proposed for providing localized treatment of bone infection. For instance, antibiotic beads have been employed that are made from bone cement (e.g., polymethylmethacrylate). Such beads may be selectively disposed in a void (e.g., surgical void) to locally deliver a bactericidal level of an antibiotic agent. While providing some benefits, these beads still have a variety of disadvantages. For example, most commercially available beads must be hand mixed with the antibiotic agent by a medical professional just prior to placement in a void. In addition to being an inefficient and time consuming process, such “hand-mixed” beads also tend to have an inconsistent level of the antibiotic agent and an increased risk of contamination.

As such, a need currently exists for improved antibiotic beads that are capable of being used to treat an infection in a wound site (e.g., bone infection).

### SUMMARY OF THE INVENTION

In accordance with one embodiment of the present invention, an antibiotic bead having a size of from about 0.5 to about 20 millimeters is disclosed. The bead comprises an antibiotic agent dispersed within a polymer matrix, the polymer matrix containing a semi-crystalline olefin copolymer. In another embodiment, a package is disclosed that comprises a plurality of antibiotic beads having a size of from about 0.5 to about 20 millimeters. The beads comprise an antibiotic agent that is dispersed within a polymer matrix, the polymer matrix containing a semi-crystalline olefin copolymer. In yet another embodiment, a method for treating an infection is disclosed that comprises disposing a plurality of beads within a wound site of a patient having a size of from about 0.5 to about 20 millimeters. The beads comprise an antibiotic agent that is dispersed within a polymer matrix, the polymer matrix containing a semi-crystalline olefin copolymer.

Other features and aspects of the present invention are set forth in greater detail below.

### DETAILED DESCRIPTION

It is to be understood by one of ordinary skill in the art that the present discussion is a description of exemplary embodiments only, and is not intended as limiting the broader aspects of the present invention.

Generally speaking, the present invention is directed to antibiotic beads can have a variety of benefits, such as inhibiting, preventing, and/or treating an infection. Typical uses of such beads may include, for instance, prophylaxis of infections for open fractures and total joint arthroplasties and in the treatment of acute and chronic osteomyelitis, periprosthetic joint infections, diabetic infections, and septic non-unions. The beads may have a variety of different cross-sectional shapes, such as generally circular, square, rectangular, ovular, elliptical, triangular, etc., as well as irregular shapes. The beads have a size (e.g., diameter) of from about 0.5 to about 20 millimeters, in some embodiments from about 1 to about 10 millimeters, and in some embodiments, from about 2 to about 6 millimeters. The beads may also have a volume from about 0.1 to about 1 cubic centimeter per bead, in some embodiments from about 0.2 to about 0.9 cubic centimeter per bead, and in some embodiments, from about 0.3 to about 0.8 cubic centimeters per bead. Regardless of their particular size and shape, the beads contain a polymer matrix that includes a semi-crystalline olefin copolymer (e.g., ethylene vinyl acetate copolymer) and in which is dispersed an effective amount of at least one antibiotic agent. Through selective control over the particular nature of the polymer matrix, the antibiotic agent(s), and the manner in which they are combined together, the present inventors have discovered that the resulting beads can be effective for treating an infection over a prolonged period of time. For example, such beads may be placed in a wound site, such as in a surgical site in which bone infection has been removed, to provide the desired degree of antibiotic treatment for a time period of about 5 days or more, in some embodiments about 10 days or more, in some embodiments from about 20 days to about 60 days, and in some embodiments, from about 30 days to about 50 days (e.g., about 40 days). Without intending to be limited by theory, it is believed that the hydrophobic nature of the olefin copolymer can slow the degree to which moisture diffuses into the matrix, which in turn, allows the antibiotic agent to be release gradually over the desired period of time.

Various embodiments of the present invention will now be described in more detail.

### I. Antibiotic Beads

A. Polymer Matrix

As indicated above, the polymer matrix contains at least one semi-crystalline olefin copolymer. The melting temperature of the olefin copolymer may, for instance, range from about 40° C. to about 140° C., in some embodiments from about 50° C. to about 125° C., and in some embodiments, from about 60° C. to about 120° C., as determined in accordance with ASTM D3418-15. Such copolymers are generally derived from at least one olefin monomer (e.g., ethylene, propylene, etc.) and at least one polar monomer that is grafted onto the polymer backbone and/or incorporated as a constituent of the polymer (e.g., block or random copolymers). Suitable polar monomers include, for instance, a vinyl acetate, vinyl alcohol, maleic anhydride, maleic acid, (meth)acrylic acid (e.g., acrylic acid, methacrylic acid, etc.), (meth)acrylate (e.g., acrylate, methacrylate, ethyl acrylate, methyl methacrylate, ethyl methacrylate, etc.), and so forth. A wide variety of such copolymers may generally be employed in the polymer composition, such as ethylene vinyl acetate copolymers, ethylene (meth)acrylic acid polymers (e.g., ethylene acrylic acid copolymers and partially neutralized ionomers of these copolymers, ethylene methacrylic acid copolymers and partially neutralized ionomers of these copolymers, etc.), ethylene (meth)acrylate polymers (e.g., ethylene methylacrylate copolymers, ethylene ethyl acrylate copolymers, ethylene butyl acrylate copolymers, etc.), and so forth. Regardless of the particular monomers selected, the present inventors have discovered that certain aspects of the copolymer can be selectively controlled to help achieve the desired release properties. For instance, the polar monomeric content of the copolymer may be selectively controlled to be within a range of from about 10 wt. % to about 45 wt. %, in some embodiments about 15 wt. % to about 43 wt. %, and in some embodiments, from about 20 wt. % to about 40 wt. %. Conversely, the olefin monomeric content of the copolymer may be likewise be within a range of from about 55 wt. % to about 90 wt. %, in some embodiments about 57 wt. % to about 85 wt. %, and in some embodiments, from about 60 wt. % to about 80 wt. %.

In one particular embodiment, for example, the polymer composition may contain an ethylene vinyl acetate polymer, which is a copolymer that is derived from at least one ethylene monomer and at least one vinyl acetate monomer. The density of the ethylene vinyl acetate copolymer may also range from about 0.900 to about 1.00 gram per cubic centimeter (g/cm³), in some embodiments from about 0.910 to about 0.980 g/cm³, and in some embodiments, from about 0.930 to about 0.960 g/cm³, as determined in accordance with ASTM D1505-10. Still further, the melt flow index of the ethylene vinyl acetate copolymer may range from about 0.1 to about 30 g/10 min, in some embodiments from about 0.5 to about 20 g/10 min, and in some embodiments, from about 1 to about 10 g/10 min, as determined in accordance with ASTM D1238-13 at a temperature of 190° C. and a load of 2.16 kilograms. Examples of suitable ethylene vinyl acetate copolymers that may be employed include those available from Celanese under the designation ATEVA® (e.g., ATEVA® 2861A or 2803W); DuPont under the designation ELVAX® (e.g., ELVAX® 265 or 260); and Arkema under the designation EVATANE® (e.g., EVATANE 28-03). Any of a variety of techniques may generally be used to form the ethylene vinyl acetate copolymer with the desired properties as is known in the art. In one embodiment, the polymer is produced by copolymerizing an ethylene monomer and a vinyl acetate monomer in a high pressure reaction. Vinyl acetate may be produced from the oxidation of butane to yield acetic anhydride and acetaldehyde, which can react together to form ethylidene diacetate. Ethylidene diacetate can then be thermally decomposed in the presence of an acid catalyst to form the vinyl acetate monomer. Examples of suitable acid catalysts include aromatic sulfonic acids (e.g., benzene sulfonic acid, toluene sulfonic acid, ethylbenzene sulfonic acid, xylene sulfonic acid, and naphthalene sulfonic acid), sulfuric acid, and alkanesulfonic acids, such as described in U.S. Pat. No. 2,425,389 to Oxley et al; U.S. Pat. No. 2,859,241 to Schnizer; and U.S. Pat. No. 4,843,170 to Isshiki et al. The vinyl acetate monomer can also be produced by reacting acetic anhydride with hydrogen in the presence of a catalyst instead of acetaldehyde. This process converts vinyl acetate directly from acetic anhydride and hydrogen without the need to produce ethylidene diacetate. In yet another embodiment, the vinyl acetate monomer can be produced from the reaction of acetaldehyde and a ketene in the presence of a suitable solid catalyst, such as a perfluorosulfonic acid resin or zeolite.

If desired, the matrix may be formed entirely from a semi-crystalline olefin copolymer or a blend of such copolymers. Of course, if desired, other biocompatible polymers may also be employed in the polymer matrix if so desired, such as polyethylene, polypropylene, acrylic acid polymers (e.g., polymethylmethacrylate), poly(lactic acid), poly(glycolic acid), poly(lactic-co-glycolic acid), poly(butylene succinate), poly(caprolactone), polyanhydrides, poly(vinyl alcohol), starches, cellulosics, chitans, chitosans, cellulose esters, cellulose acetate, nitrocellulose, etc. When employed, however, such additional polymers typically constitute no more than about 20 wt. %, in some embodiments no more than about 10 wt. %, and in some embodiments, from about 0.01 wt. % to about 5 wt. % of the polymer matrix.

B. Antibiotic Agent

Any of a variety of antibiotic agents may generally be employed in the beads of the present invention. Typically, it is desired that the antibiotic agent(s) employed in the beads are generally hydrophilic in nature and also effective against a broad spectrum of bacteria, such as Gram-negative bacteria (e.g., Pseudomonas, Proteus, E. coli, K. pneumoniae, Enterobacter aerogenes, Serratia, etc.) and Gram-positive (e.g., S. Aureus, etc.). It is also generally desirable that the antibiotic agent(s) remain stable at high temperatures so that they can be incorporated into the polymer matrix at or near the melting temperature of the olefin copolymer. For example, the antibiotic agent(s) typically remain stable at temperatures of from about 25° C. to about 140° C., in some embodiments from about 40° C. to about 140° C., in some embodiments from about 40° C. to about 120° C., and in some embodiments, from about 50° C. to about 100° C. Particularly suitable hydrophilic, heat-stable antibiotic agents for treating bone infection may include, for instance, vancomycin (glycopeptide), gentamycin (am inoglycoside), tobramycin (am inoglycoside), doxycycline (tetracycline), minocycline (tetracycline), etc., as well as combinations thereof. Of course, other antibiotic agents may also be employed, such as tetracycline, amoxicillin, amoxicillin/clavulanic acid, penicillin, metronidazole, clindamycine, chlortetracycline, demeclocycline, oxytetracycline, amikacin, gentamicin, kanamycin, neomycin, netilmicin, streptomycin, tobramycin, cefadroxil, cefazolin, cephalexin, cephalothin, cephapirin, cephradine, cefaclor, cefamandole, cefametazole, cefonicid, cefotetan, cefoxitine, cefpodoxime, cefprozil, cefuroxime, cefdinir, cefixime, cefoperazone, cefotaxime, ceftazidime, ceftibuten, ceftizoxime, ceftriaxone, cefepime, azithromycin, clarithromycin, dirithromycin, erythromycin, lincomycin, troleandomycin, bacampicillin, carbenicillin, cloxacillin, dicloxacillin, meticillin, mezlocillin, nafcillin, oxacillin, piperacillin, ticarcillin, cinoxacin, ciprofloxacin, enoxacin, grepafloxacin, levofloxacin, lomefloxacin, nalidixic acid, norfloxacin, ofloxacin, sparfloxacin, sulfisoxazole, sulfacytine, sulfadiazine, sulfamethoxazole, sulfisoxazole, dapson, aztreonam, bacitracin, capreomycin, chloramphenicol, clofazimine, colistimethate, colistin, cycloserine, fosfomycin, furazolidone, methenamine, nitrofurantoin, pentamidine, rifabutin, rifampin, spectinomycin, trimethoprim, trimetrexate glucuronate, etc., as well as combinations thereof.

The dosage level of the antibiotic agents employed in the beads may vary depending on the particular antibiotic agent employed and the time period for which it is intend to be released. Typically, however, antibiotic agents are present in an amount of from about 1 wt. % to about 20 wt. %, in some embodiments from about 2 wt. % about 15 wt. %, and in some embodiments, from about 4 wt. % to about 12 wt. % of a bead. Likewise, the polymer matrix typically constitutes from about 80 wt. % to about 99 wt. %, in some embodiments from about 85 wt. % to about 98 wt. %, and in some embodiments, from about 88 wt. % to about 96 wt. % of a bead.

C. Optional Excipients

The beads may optionally contain one or more excipients if so desired, such as radiocontrast agents, release modifiers, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 20 wt. %, and in some embodiments, from about 0.05 wt. % to about 15 wt. %, and in some embodiments, from about 0.1 wt. % to about 10 wt. % of the beads. In one embodiment, for instance, a radiocontrast agent may be employed to help ensure that the beads can be detected in an X-ray based imaging technique (e.g., computed tomography, projectional radiography, fluoroscopy, etc.). Examples of such agents include, for instance, barium-based compounds, iodine-based compounds, zirconium-based compounds (e.g., zirconium dioxide), etc. One particular example of such an agent is barium sulfate. Other known antimicrobial agents and/or preservatives may also be employed to help prevent surface growth and attachment of bacteria, such as metal compounds (e.g., silver, copper, or zinc), metal salts, quaternary ammonium compounds, etc.

A release modifier may also be incorporated into the beads to help facilitate the release of the antibiotic agent(s) from the beads during use. When employed, such release modifiers(s) typically constitute from about 0.5 wt. % to about 20 wt. %, and in some embodiments, from about 1 wt. % to about 15 wt. %, and in some embodiments, from about 4 wt. % to about 10 wt. % of the beads. One example of such a release modifier is a hydrophilic polymer that is soluble and/or swellable in water. Examples of such hydrophilic polymers include, for instance, sodium, potassium and calcium alginates, carboxymethylcellulose, agar, gelatin, polyvinyl alcohols, polyalkylene glycols, collagen, pectin, chitin, chitosan, poly-1-caprolactone, polyvinylpyrrolidone, polysaccharides, hydrophilic polyurethane, polyhydroxyacrylate, dextran, xanthan, hydroxypropyl cellulose, methyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, acrylic acid, and combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

To help create a uniform dispersion of the antibiotic agent(s) and polymer matrix, one or more nonionic, anionic, and/or amphoteric surfactants may also be employed in certain embodiments. When employed, such surfactant(s) typically constitute from about 0.05 wt. % to about 8 wt. %, and in some embodiments, from about 0.1 wt. % to about 6 wt. %, and in some embodiments, from about 0.5 wt. % to about 3 wt. % of the beads. Nonionic surfactants, which typically have a hydrophobic base (e.g., long chain alkyl group or an alkylated aryl group) and a hydrophilic chain (e.g., chain containing ethoxy and/or propoxy moieties), are particularly suitable. Some suitable nonionic surfactants that may be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, condensation products of ethylene oxide with alcohols, fatty acid esters, monoglyceride or diglycerides of long chain alcohols, and mixtures thereof. Particularly suitable nonionic surfactants may include ethylene oxide condensates of fatty alcohols, polyoxyethylene ethers of fatty acids, polyoxyethylene sorbitan fatty acid esters, and sorbitan fatty acid esters, etc. The fatty components used to form such emulsifiers may be saturated or unsaturated, substituted or unsubstituted, and may contain from 6 to 22 carbon atoms, in some embodiments from 8 to 18 carbon atoms, and in some embodiments, from 12 to 14 carbon atoms. Sorbitan fatty acid esters (e.g., monoesters, diester, triesters, etc.) that have been modified with polyoxyethylene are one particularly useful group of nonionic surfactants. These materials are typically prepared through the addition of ethylene oxide to a 1,4-sorbitan ester. The addition of polyoxyethylene converts the lipophilic sorbitan ester surfactant to a hydrophilic surfactant that is generally soluble or dispersible in water. Such materials are commercially available under the designation TWEEN® (e.g., TWEEN® 80, or polyethylene (20) sorbitan monooleate).

Regardless of the particular components employed, the beads may be formed through a variety of known techniques, such as by hot-melt extrusion, injection molding, solvent casting, dip coating, spray coating, microextrusion, coacervation, etc. In one embodiment, a hot-melt extrusion technique may be employed. Hot-melt extrusion is generally a solvent-free process in which the components of the beads (e.g., semi-crystalline olefin copolymer, antibiotic agent(s), etc.) may be melt blended and optionally shaped in a continuous manufacturing process to enable consistent output quality at high throughput rates. This technique is particularly well suited to olefin copolymers. Namely, such copolymers typically exhibit a relatively high degree of long-chain branching with a broad molecular weight distribution. This combination of traits can lead to shear thinning of the copolymer during the extrusion process, which help facilitates hot-melt extrusion. Furthermore, the polar comonomer units (e.g., vinyl acetate) can serve as an “internal” plasticizer by inhibiting crystallization of the polyethylene chain segments. This may lead to a lower melting point of the olefin copolymer, which improves the overall flexibility of the resulting material and enhances its ability to be formed into beads of a wide variety of shapes and sizes.

During a hot-melt extrusion process, melt blending may occur at a temperature range of from about 40° C. to about 200° C., in some embodiments, from about 60° C. to about 180° C., and in some embodiments, from about 80° C. to about 150° C. to form a polymer composition. Any of a variety of melt blending techniques may generally be employed. For example, the components (e.g., semi-crystalline olefin copolymer, antibiotic agent, and other optional excipients) may be supplied separately or in combination to an extruder that includes at least one screw rotatably mounted and received within a barrel (e.g., cylindrical barrel). The extruder may be a single screw or twin screw extruder. For example, one embodiment of a single screw extruder may contain a housing or barrel and a screw rotatably driven on one end by a suitable drive (typically including a motor and gearbox). If desired, a twin-screw extruder may be employed that contains two separate screws. The configuration of the screw is not particularly critical and it may contain any number and/or orientation of threads and channels as is known in the art. For example, the screw typically contains a thread that forms a generally helical channel radially extending around a core of the screw. A feed section and melt section may be defined along the length of the screw. The feed section is the input portion of the barrel where the olefin copolymer(s) and/or antibiotic agent(s) are added. The melt section is the phase change section in which the copolymer is changed from a solid to a liquid-like state. While there is no precisely defined delineation of these sections when the extruder is manufactured, it is well within the ordinary skill of those in this art to reliably identify the feed section and the melt section in which phase change from solid to liquid is occurring. Although not necessarily required, the extruder may also have a mixing section that is located adjacent to the output end of the barrel and downstream from the melting section. If desired, one or more distributive and/or dispersive mixing elements may be employed within the mixing and/or melting sections of the extruder. Suitable distributive mixers for single screw extruders may include, for instance, Saxon, Dulmage, Cavity Transfer mixers, etc. Likewise, suitable dispersive mixers may include Blister ring, Leroy/Maddock, CRD mixers, etc. As is well known in the art, the mixing may be further improved by using pins in the barrel that create a folding and reorientation of the polymer melt, such as those used in Buss Kneader extruders, Cavity Transfer mixers, and Vortex Intermeshing Pin mixers.

If desired, the ratio of the length (“L”) to diameter (“D”) of the screw may be selected to achieve an optimum balance between throughput and blending of the components. The L/D value may, for instance, range from about 10 to about 50, in some embodiments from about 15 to about 45, and in some embodiments from about 20 to about 40. The length of the screw may, for instance, range from about 0.1 to about 5 meters, in some embodiments from about 0.4 to about 4 meters, and in some embodiments, from about 0.5 to about 2 meters. The diameter of the screw may likewise be from about 5 to about 150 millimeters, in some embodiments from about 10 to about 120 millimeters, and in some embodiments, from about 20 to about 80 millimeters. In addition to the length and diameter, other aspects of the extruder may also be selected to help achieve the desired degree of blending. For example, the speed of the screw may be selected to achieve the desired residence time, shear rate, melt processing temperature, etc. For example, the screw speed may range from about 10 to about 800 revolutions per minute (“rpm”), in some embodiments from about 20 to about 500 rpm, and in some embodiments, from about 30 to about 400 rpm. The apparent shear rate during melt blending may also range from about 100 seconds⁻¹ to about 10,000 seconds⁻¹, in some embodiments from about 500 seconds⁻¹ to about 5000 seconds⁻¹, and in some embodiments, from about 800 seconds⁻¹ to about 1200 seconds⁻¹. The apparent shear rate is equal to 4Q/πR³, where Q is the volumetric flow rate (“m³/s”) of the polymer melt and R is the radius (“m”) of the capillary (e.g., extruder die) through which the melted polymer flows.

Once melt blended together, the resulting polymer composition may be in the form of pellets, sheets, fibers, filaments, etc., which may be shaped into a bead using a variety of known shaping techniques, such as injection molding, compression molding, nanomolding, overmolding, blow molding, three-dimensional printing, etc. Injection molding may, for example, occur in two main phases—i.e., an injection phase and holding phase. During the injection phase, a mold cavity is filled with the molten polymer composition. The holding phase is initiated after completion of the injection phase in which the holding pressure is controlled to pack additional material into the cavity and compensate for volumetric shrinkage that occurs during cooling. After the shot has built, it can then be cooled. Once cooling is complete, the molding cycle is completed when the mold opens and the part is ejected, such as with the assistance of ejector pins within the mold. Any suitable injection molding equipment may generally be employed in the present invention. In one embodiment, an injection molding apparatus may be employed that includes a first mold base and a second mold base, which together define a mold cavity having the shape of a bead. The molding apparatus includes a resin flow path that extends from an outer exterior surface of the first mold half through a sprue to a mold cavity. The polymer composition may be supplied to the resin flow path using a variety of techniques. For example, the composition may be supplied (e.g., in the form of pellets) to a feed hopper attached to an extruder barrel that contains a rotating screw (not shown). As the screw rotates, the pellets are moved forward and undergo pressure and friction, which generates heat to melt the pellets. A cooling mechanism may also be provided to solidify the resin into the shape of beads within the mold cavity. For instance, the mold bases may include one or more cooling lines through which a cooling medium flows to impart the desired mold temperature to the surface of the mold bases for solidifying the molten material. The mold temperature (e.g., temperature of a surface of the mold) may range from about 50° C. to about 120° C., in some embodiments from about 60° C. to about 110° C., and in some embodiments, from about 70° C. to about 90° C.

As indicated above, another suitable technique for forming beads of the desired shape and size is three-dimensional printing. During this process, the polymer composition may be incorporated into a printer cartridge that is readily adapted for use with a printer system. The printer cartridge may, for example, contains a spool or other similar device that carries the polymer composition. When supplied in the form of filaments, for example, the spool may have a generally cylindrical rim about which the filaments are wound. The spool may likewise define a bore or spindle that allows it to be readily mounted to the printer during use. Any of a variety of three-dimensional printer systems can be employed in the present invention. Particularly suitable printer systems are extrusion-based systems, which are often referred to as “fused deposition modeling” systems. For example, the polymer composition may be supplied to a build chamber of a print head that contains a platen and gantry. The platen may move along a vertical z-axis based on signals provided from a computer-operated controller. The gantry is a guide rail system that may be configured to move the print head in a horizontal x-y plane within the build chamber based on signals provided from controller. The print head is supported by the gantry and is configured for printing the build structure on the platen in a layer-by-layer manner, based on signals provided from the controller. For example, the print head may be a dual-tip extrusion head.

### II. Treatment of Infection

The antibiotic beads of the present invention may be used in a variety of different ways to treat infection in a wound site of a patient (e.g., human, animal, etc.). For example, the beads may be selectively disposed in a void in which infectious tissue (e.g., bone) has been surgically removed due to chronic infection. The beads may also be employed at a site of a bone fracture, placement of metal rods, plates or metallic fixators and joint replacement devices, or placement of graft materials used in cardiovascular, general, gynecologic, and neurosurgical procedures. The beads may be implanted, injected, or otherwise placed completely or partially within the wound site. Any number of beads may be employed for treating infection. In certain embodiments, for instance, the number of beads employed may be from 2 to 150, in some embodiments from 5 to 100, in some embodiments from 10 to 80, and in some embodiments, from 20 to 60. The beads may be packed within the wound site so that some or all of the beads are in contact with adjacent beads. Alternatively, certain beads may also be spaced apart by a certain distance, such as by about 2 to about 15 millimeters, and in some embodiments, from about 3 to about 10 millimeters. The beads may be independently disposed within the wound site, or they may also be connected together through a string, strand, or other linking material. The beads may contain the same or different types of antibiotic agents. For instance, one type of bacteria may be initially present in a wound site, but as treatment with a first antibiotic eliminates such bacteria, a second type of bacteria may become more prevalent. In such cases, it may be desirable to employ a plurality of first beads that contain a first antibiotic agent effective against one type of bacteria and a plurality of second beads containing a second antibiotic agent effective against another type of bacteria. Likewise, the beads may also contain the same or different concentration of antibiotic agent(s). In one embodiment, for instance, a plurality of first beads may be employed that contain an antibiotic agent at a first concentration and a plurality of second beads may be employed that contain the same or different antibiotic agent, but in a second concentration greater than the first concentration employed for the first beads. In this manner, the first and second beads may exhibit a different release profile over an extended period of time, which can help provide optimum level of infection control in the wound site.

If desired, a single bead or a plurality of beads may be sealed within a package (e.g., sterile blister package) prior to use. The materials and manner in which the package is sealed may vary as is known in the art. In one embodiment, for instance, the package may contain a substrate that includes any number of layers desired to achieve the desired level of protective properties, such as 1 or more, in some embodiments from 1 to 4 layers, and in some embodiments, from 1 to 3 layers. Typically, the substrate contains a polymer film, such as those formed from a polyolefin (e.g., ethylene copolymers, propylene copolymers, propylene homopolymers, etc.), polyester (e.g., polyethylene terephthalate, polyethylene naphthalate, polybutylene terephthalate, etc.), vinyl chloride polymer, vinyl chloridine polymer, ionomer, etc., as well as combinations thereof. One or multiple panels of the film may be sealed together (e.g., heat sealed), such as at the peripheral edges, to form a cavity within which the antibiotic beads may be stored. For example, a single film may be folded at one or more points and sealed along its periphery to define the cavity within with the beads are located. To treat an infection, the package may be opened, such as by breaking the seal, and the beads may then be removed and placed in a wound site.

These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. An antibiotic bead having a size of from about 0.5 to about 20 millimeters, wherein the bead comprises an antibiotic agent dispersed within a polymer matrix, the polymer matrix containing a semi-crystalline olefin copolymer.

2. The antibiotic bead of claim 1, wherein the bead has a generally circular cross-sectional shape.

3. The antibiotic bead of claim 1, wherein the semi-crystalline olefin copolymer is derived from at least one olefin monomer and at least one polar monomer.

4. The antibiotic bead of claim 3, wherein the olefin monomer includes ethylene.

5. The antibiotic bead of claim 3, wherein the polar monomer includes vinyl acetate, vinyl alcohol, maleic anhydride, maleic acid, acrylic acid, methacrylic acid, acrylate, methacrylate, ethyl acrylate, methyl methacrylate, ethyl methacrylate, or a combination thereof.

6. The antibiotic bead of claim 5, wherein the polar monomer constitutes from about 10 wt. % to about 45 wt. % of the copolymer.

7. The antibiotic bead of claim 1, wherein the olefin copolymer has a melting temperature of from about 40° C. to about 140° C. as determined in accordance with ASTM D3418-15.

8. The antibiotic bead of claim 1, wherein the olefin copolymer includes an ethylene vinyl acetate copolymer.

9. The antibiotic bead of claim 8, wherein the ethylene vinyl acetate copolymer has a melt flow index of from about 0.1 to about 30 grams per 10 minutes as determined in accordance with ASTM D1238-13 at a temperature of 190° C. and a load of 2.16 kilograms.

10. The antibiotic bead of claim 1, wherein the polymer matrix is formed entirely from semi-crystalline olefin copolymers.

11. The antibiotic bead of claim 1, wherein the antibiotic agent is stable at a temperature of from about 40° C. to about 120° C.

12. The antibiotic bead of claim 1, wherein the antibiotic agent includes vancomycin, gentamycin, tobramycin, doxycycline, minocycline, or a combination thereof.

13. The antibiotic bead of claim 1, wherein antibiotic agents are present in an amount of from about 1 wt. % to about 20 wt. % of the bead.

14. The antibiotic bead of claim 1, wherein the bead further comprises a hydrophilic polymer.

15. The antibiotic bead of claim 14, wherein the hydrophilic polymer includes a polyalkylene glycol.

16. The antibiotic bead of claim 1, wherein the bead further comprises a nonionic surfactant.

17. The antibiotic bead of claim 1, wherein the bead further comprises a radiocontrast agent.

18. The antibiotic bead of claim 1, wherein the bead further comprises a colorizing agent.

19. The antibiotic bead of claim 1, wherein the bead further comprises an antimicrobial agent and/or preservative.

20. A method for forming the antibiotic bead of claim 1, the method comprising:
melt blending the semi-crystalline olefin copolymer and the antibiotic agent within an extruder to form a polymer composition; and
shaping the polymer composition into the form of a bead.

21. The method of claim 20, wherein melt blending occurs at a temperature range of from about 40° C. to about 200° C.

22. The method of claim 20, wherein shaping of the polymer composition includes injecting the composition into a mold cavity and allowing the composition to solidify therein to form the bead.

23. The method of claim 20, wherein shaping of the polymer composition includes printing the composition to form the bead.

24. A method for treating an infection, the method comprising disposing the bead of claim 1 within a wound site of a patient.

25. The method of claim 24, wherein the bead is allowed to remain within the wound site for a time period of about 5 days or more.

26. The method of claim 24, wherein the wound site includes a void in which infectious bone has been surgically removed, and wherein the bead is disposed within the void.

27. A package comprising a plurality of antibiotic beads having a size of from about 0.5 to about 20 millimeters, wherein the beads comprise an antibiotic agent that is dispersed within a polymer matrix, the polymer matrix containing a semi-crystalline olefin copolymer.

28. The package of claim 27, wherein the beads have a generally circular cross-sectional shape.

29. The package of claim 27, wherein the semi-crystalline olefin copolymer is derived from at least one olefin monomer and at least one polar monomer.

30. The package of claim 27, wherein the olefin monomer includes ethylene.

31. The package of claim 27, wherein the polar monomer includes vinyl acetate, vinyl alcohol, maleic anhydride, maleic acid, acrylic acid, methacrylic acid, acrylate, methacrylate, ethyl acrylate, methyl methacrylate, ethyl methacrylate, or a combination thereof.

32. The package of claim 27, wherein the polar monomer constitutes from about 10 wt. % to about 45 wt. % of the copolymer.

33. The package of claim 27, wherein the olefin copolymer has a melting temperature of from about 40° C. to about 140° C. as determined in accordance with ASTM D3418-15.

34. The package of claim 27, wherein the olefin copolymer includes an ethylene vinyl acetate copolymer.

35. The package of claim 34, wherein the ethylene vinyl acetate copolymer has a melt flow index of from about 0.1 to about 30 grams per 10 minutes as determined in accordance with ASTM D1238-13 at a temperature of 190° C. and a load of 2.16 kilograms.

36. The package of claim 27, wherein the antibiotic agent includes vancomycin, gentamycin, tobramycin, doxycycline, minocycline, or a combination thereof.

37. The package of claim 27, wherein the package contains from 2 to 150 antibiotic beads.

38. The package of claim 27, wherein the package contains a plurality of first beads containing a first antibiotic agent and a plurality of second beads containing a second antibiotic agent, wherein the first antibiotic agent is different than the second antibiotic agent.

39. The package of claim 27, wherein the package contains a plurality of first beads containing an antibiotic agent at a first concentration and a plurality of second beads containing an antibiotic agent at a second concentration, wherein the second concentration is greater than the first concentration.

40. A method for treating an infection, the method comprising disposing a plurality of beads within a wound site of a patient that have a size of from about 0.5 to about 20 millimeters, wherein the beads comprise an antibiotic agent that is dispersed within a polymer matrix, the polymer matrix containing a semi-crystalline olefin copolymer.

41. The method of claim 40, wherein the beads are allowed to remain within the wound site for a time period of about 5 days or more.

42. The method of claim 40, wherein the wound site includes a void in which infectious bone has been surgically removed, and wherein the beads are disposed within the void.

43. The method of claim 40, wherein the beads have a generally circular cross-sectional shape.

44. The method of claim 40, wherein the semi-crystalline olefin copolymer is derived from at least one olefin monomer and at least one polar monomer.

45. The method of claim 44, wherein the olefin monomer includes ethylene.

46. The method of claim 44, wherein the polar monomer includes vinyl acetate, vinyl alcohol, maleic anhydride, maleic acid, acrylic acid, methacrylic acid, acrylate, methacrylate, ethyl acrylate, methyl methacrylate, ethyl methacrylate, or a combination thereof.

47. The method of claim 44, wherein the polar monomer constitutes from about 10 wt. % to about 45 wt. % of the copolymer.

48. The method of claim 40, wherein the olefin copolymer has a melting temperature of from about 40° C. to about 140° C. as determined in accordance with ASTM D3418-15.

49. The method of claim 40, wherein the olefin copolymer includes an ethylene vinyl acetate copolymer.

50. The method of claim 49, wherein the ethylene vinyl acetate copolymer has a melt flow index of from about 0.1 to about 30 grams per 10 minutes as determined in accordance with ASTM D1238-13 at a temperature of 190° C. and a load of 2.16 kilograms.

51. The method of claim 40, wherein the antibiotic agent includes vancomycin, gentamycin, tobramycin, doxycycline, minocycline, or a combination thereof.

52. The method of claim 40, wherein from 2 to 150 antibiotic beads are disposed within the wound site.

53. The method of claim 40, wherein a plurality of first beads contain a first antibiotic agent and a plurality of second beads contain a second antibiotic agent, wherein the first antibiotic agent is different than the second antibiotic agent.

54. The method of claim 40, wherein a plurality of first beads contain an antibiotic agent at a first concentration and a plurality of second beads contain an antibiotic agent at a second concentration, wherein the second concentration is greater than the first concentration.
